(19) United States
(12) Patent Application Publication
(10) Pub. No.: US 2017/0121188 A1
(43) Pub. Date: May 4, 2017

(54) LAMP CONNECTOR ASSEMBLY FOR ULTRAVIOLET WATER TREATMENT VESSELS

(71) Applicants: Paul ROPIC, Erlanger, KY (US); Jacqueline R. LAKES, Erlanger, KY (US); Berson Milieutechniek BV, Nuenen (NL)

(72) Inventors: Paul Ropic, Walton, KY (US); Jacqueline R. Lakes, Indenpendence, KY (US)

(73) Assignee: Berson Milieutechniek BV, Nuenen (NL)

(21) Appl. No.: 15/314,532

(22) PCT Filed: Jun. 5, 2015
(86) PCT No.: PCT/US15/34443

§ 371 Date: Nov. 29, 2016

Related U.S. Application Data
(60) Provisional application No. 62/008,958, filed on Jun. 6, 2014.

(51) Int. Cl. C02F 1/32

(52) U.S. Cl. CPC C02F 1/32; C02F 2303/04; C02F 2201/3227

(57) ABSTRACT

A lamp connector assembly for an ultraviolet (UV) water treatment vessel includes a sealing collar configured to connect to a headplate of the UV treatment vessel. The sealing collar includes a first lamp collar interlock feature. A lamp collar is configured to releasably connect to the sealing collar. A release member is moveably connected to the lamp collar. The release member has a locked configuration that engages the first lamp interlock feature to prevent movement of the lamp collar relative to the sealing collar and an unlocked configuration that disengages the first lamp interlock feature to allow movement of the lamp collar relative to the sealing collar.

### CROSS-REFERENCE TO RELATED APPLICATIONS

This application claims priority to U.S. Provisional Application Ser. No. 62/008,958, filed Jun. 6, 2014, entitled LAMP CONNECTOR ASSEMBLY FOR UV WATER TREATMENT VESSELS, the details of which are incorporated by reference herein.

### TECHNICAL FIELD

The present disclosure relates to ultraviolet water treatment vessels and, more particularly, to water treatment vessels that include a lamp connector assembly that safeguards user access to an ultraviolet lamp connected thereto.

### BACKGROUND

Ultraviolet (UV) radiation may be used in purification systems for use in disinfection of water or other fluids. The UV radiation acts to deactivate or kill bacteria, germs and microorganisms. The purification systems may utilize a UV lamp that is connected to a power source using a lamp mounting assembly. The lamp mounting assembly may include a socket into which the UV lamp is plugged. There is a need to protect the operator or other personnel from exposure to UV light. There is a need to protect the operator during high fluid pressure situations if a quartz sleeve is broken. There is also a need for purification systems that provide enhanced efficacy (e.g., enhanced water disinfecting capability) and lower operational costs.

### SUMMARY

Embodiments described herein are directed to lamp connector assemblies that can reduce unintended operator exposure to UV light, which can protect the operator from overexposure to UV radiation. The lamp connector assemblies inhibit removal of the UV lamp while connected to a power source due to the lamp connector assembly configuration, thereby requiring disconnection of the power source before removal of the UV lamp. The lamp connector assemblies can also protect the operator from a broken quartz sleeve when the vessel is operating with a high fluid pressure due to an increase in pressure on the lamp connector assemblies. Due to the configuration of the lamp connector assemblies, the lamp connector assemblies may not be removed until internal pressure on the lamp connector assemblies is reduced or eliminated.

In an embodiment, a lamp connector assembly for an ultraviolet (UV) water treatment vessel includes a sealing collar configured to connect to a headplate of the UV treatment vessel. The sealing collar includes a first lamp collar interlock feature. A lamp collar is configured to releasably connect to the sealing collar. A release member is moveably connected to the lamp collar. The release member has a locked configuration that engages the first lamp interlock feature to prevent movement of the lamp collar relative to the sealing collar and an unlocked configuration that disengages the first lamp interlock feature to allow movement of the lamp collar relative to the sealing collar.

In another embodiment, an ultraviolet (UV) treatment vessel includes a flow control structure including an inlet port for ingress of untreated water into a treatment chamber and an outlet port for egress of treated water from the treatment chamber. A lamp connector assembly is connected to a headplate of the flow control structure. The lamp connector assembly includes a sealing collar connected to the headplate of the UV treatment vessel. The sealing collar includes a first lamp collar interlock feature. A lamp collar is configured to releasably connect to the sealing collar. A release member is moveably connected to the lamp collar. The release member has a locked configuration that engages the first lamp interlock feature to prevent movement of the lamp collar relative to the sealing collar and an unlocked configuration that disengages the first lamp interlock feature to allow movement of the lamp collar relative to the sealing collar.

In another embodiment, a lamp connector assembly for an ultraviolet (UV) water treatment vessel includes a sealing collar that is configured to connect to a headplate of the UV treatment vessel. The sealing collar includes a first lamp collar interlock feature. A lamp collar is configured to releasably connect to the sealing collar. The first lamp collar interlock feature inhibits rotation of the lamp collar relative to the sealing collar in a locked configuration. The sealing collar includes a second interlock feature that includes an interlocking slot feature that matingly receives a locking blade of the lamp collar therein with the release member in the locked configuration to inhibit axial movement of the lamp collar away from the sealing collar.

In another embodiment, a method of safeguarding user access to an ultraviolet (UV) lamp using a lamp connector assembly is provided. The method includes providing a sealing collar configured to connect to a headplate of the UV treatment vessel. The sealing collar includes a first lamp collar interlock feature. A lamp collar is provided that is configured to releasably connect to the sealing collar. A release member is moveably connected to the lamp collar. The release member has a locked configuration that engages the first lamp interlock feature to prevent movement of the lamp collar relative to the sealing collar and an unlocked configuration that disengages the first lamp interlock feature to allow movement of the lamp collar relative to the sealing collar.

### BRIEF DESCRIPTION OF THE DRAWINGS

The following description of specific embodiments of the present disclosure can be best understood when read in conjunction with the following drawings, where like structure is indicated with like reference numerals and in which:

FIG. 1 is a diagrammatic illustration of a UV treatment vessel incorporating concepts of the present disclosure;

FIG. 2 is an end view of the UV treatment vessel of FIG. 1 illustrating lamp connector assemblies incorporating concepts of the present disclosure;

FIG. 3 is a section view of a headplate of the UV treatment vessel of FIG. 2 incorporating concepts of the present disclosure;

FIG. 4 is a side, perspective view of one of the lamp connector assemblies of FIG. 2 in a locked configuration incorporating concepts of the present disclosure;

FIG. 5 is another side, perspective view of the lamp connector assembly of FIG. 4 in a locked configuration incorporating concepts of the present disclosure;

FIG. 6 is a side view of the lamp connector assembly of FIG. 4 in an unlocked configuration incorporating concepts of the present disclosure;

FIG. 7 is an exploded view of the lamp connector assembly of FIG. 4 incorporating concepts of the present disclosure;

FIG. 8 is a side, perspective view of a sealing collar in isolation incorporating concepts of the present disclosure;

FIG. 9 is another side, perspective view of the sealing collar of FIG. 8 incorporating concepts of the present disclosure;

FIG. 10 is a bottom, perspective of a lamp collar in isolation incorporating concepts of the present disclosure;

FIG. 11 is a top, perspective view of a release cap member in isolation incorporating concepts of the present disclosure;

FIG. 12 is a section view of the lamp connector assembly of FIG. 4 in a locked configuration incorporating concepts of the present disclosure;

FIGS. 13-15 illustrate operation of the lamp connector assembly of FIG. 4 by moving the release cap member from the locked configuration to the unlocked configuration incorporating concepts of the present disclosure;

FIG. 16 illustrates removal of the lamp collar from the sealing collar incorporating concepts of the present disclosure;

FIG. 17 illustrates another lamp connector assembly incorporating concepts of the present disclosure;

FIG. 18 illustrates a more detailed view of the lamp connector assembly of FIG. 17 in an unlocked configuration incorporating concepts of the present disclosure;

FIG. 19 illustrates the lamp connector assembly of FIG. 18 in a locked configuration incorporating concepts of the present disclosure;

FIG. 20 illustrates the lamp collar released from the sealing collar of the lamp connector assembly of FIG. 17 incorporating concepts of the present disclosure;

FIG. 21 is an elevated, perspective view of the lamp collar of the lamp connector assembly of FIG. 17 incorporating concepts of the present disclosure;

FIG. 22 illustrates a section view of the lamp connector assembly of FIG. 17 in a locked configuration incorporating concepts of the present disclosure;

FIG. 23 illustrates another view of the lamp connector assembly in the locked configuration incorporating concepts of the present disclosure;

FIG. 24 illustrates a release knob member of the lamp connector assembly of FIG. 17 received within an aperture of a ramp member incorporating concepts of the present disclosure;

FIG. 25 illustrates a detailed, side view of the ramp member incorporating concepts of the present disclosure; and

FIG. 26 illustrates the lamp connector assembly of FIG. 17 connected to a headpiece of a UV treatment vessel incorporating concepts of the present disclosure.

### DETAILED DESCRIPTION

Embodiments described herein generally relate to UV water treatment vessels for treating a fluid such as water. The water treatment vessels may include a large stainless steel pipe or other flow control structure with one or more UV purification lamps mounted therein. As will be described in greater detail below, the UV water treatment vessels may be provided with a lamp connector assembly that safeguards user access to a UV lamp connected thereto. Such a lamp connector assembly can inhibit disassembly of the lamp connector assembly under certain conditions, such as when the UV lamp is connected to a power source and/or when the UV lamp connector is under certain pressure conditions.

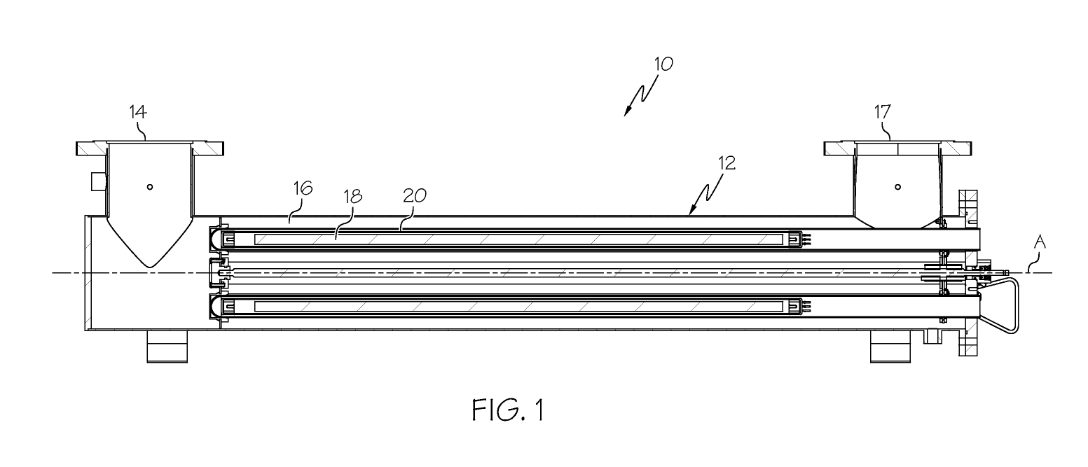

Referring to FIG. 1, an embodiment of a UV water treatment vessel 10 includes a flow control structure 12 (e.g., a stainless steel pipe or vessel) including an inlet port 14 for ingress of untreated water into a treatment chamber 16 and an outlet port 17 for egress of treated water from the treatment chamber 16. An elongated axis A extends along the length of the flow control structure 12. A UV lamp assembly 18 including a UV lamp is located in the flow control structure 12, with the UV lamp separated from the treatment chamber 16 by a sleeve 20. In some embodiments, one or more UV lamps may be isolated from direct exposure to the water by one or more quartz sleeves 20. The sleeve 20 allows the UV lamp to operate optimally and also allows operators to replace the UV lamp without draining the vessel. Water that contains high levels of organics or other contaminants may require being chambered with automated internal monitoring and cleaning mechanisms.

Although there are a variety of types of UV lamps that can be used in the UV treatment chamber 16, the orientation aspect of the present disclosure may utilize amalgam lamps. These lamps are called amalgam lamps because they contain solid amalgam. The amalgam is typically an alloy of mercury with another element, such as indium or gallium, and function to control the mercury vapor pressure of the lamp.

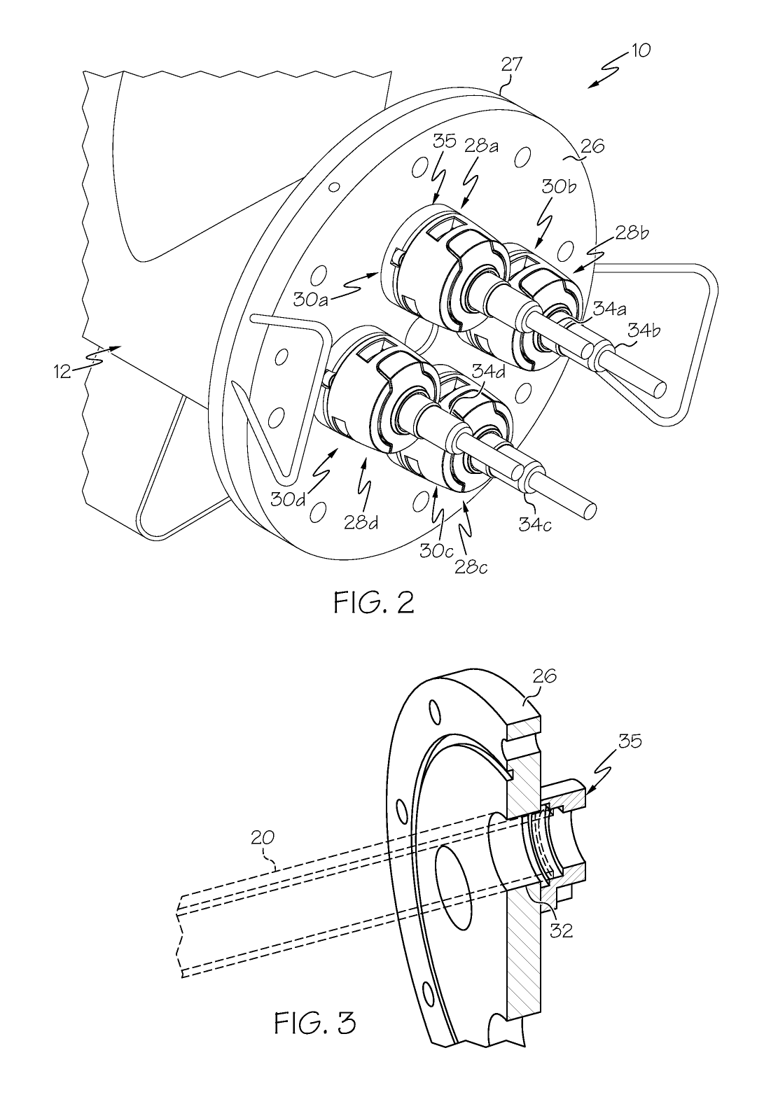

Referring to FIG. 2, the water treatment vessel 10 includes the flow control structure 12 including a front headplate 26 connected thereto at an end 27 of the flow control structure 12. The front headplate 26 includes one or more lamp connector locations 28 where one or more lamp connector assemblies 30 may be connected to the front headplate 26. In the illustrated embodiment, the front headplate 26 includes four lamp connector locations 28a-28d and four associated lamp connector assemblies 30a-30d. In other embodiments, the front headplate 26 may include more or less than four lamp connector locations 28a-28d and four lamp connector assemblies 30a-30d. The lamp connector locations 28a-28d each include an opening 32 through the front headplate 26 to allow for connection of a UV lamp to each of the lamp connector assemblies 30a-30d (FIG. 3). Electrical connectors 34a-34d are connected to the lamp connector assemblies 30a-30d for electrical connection with the UV lamps. FIG. 3 illustrates the opening 32 through the front headplate 26. A sealing collar 35 (sometimes referred to as a quartz collar) sealingly connects the sleeve 20 (sometimes referred to as a quartz sleeve) to the front headplate 26 thereby providing a water-tight seal between the sealing collar 35 and the front headplate 26.

The UV lamps are used to disinfect water, waste water, reuse water, process water, or other fluids. Untreated water is gravity controlled or pumped under pressure through the water treatment vessel 10 with the one or more UV lamps. The water pressure may typically be up to 150 pounds per square inch (psi). Higher pressures are possible. Throughout the operation of the water treatment vessel 10, the UV lamps need to be removed from the water treatment vessel 10 at regular intervals for inspection and service. In order to remove the UV lamps, the lamp connector assemblies 30a-30d are disassembled to gain access to the UV lamps.

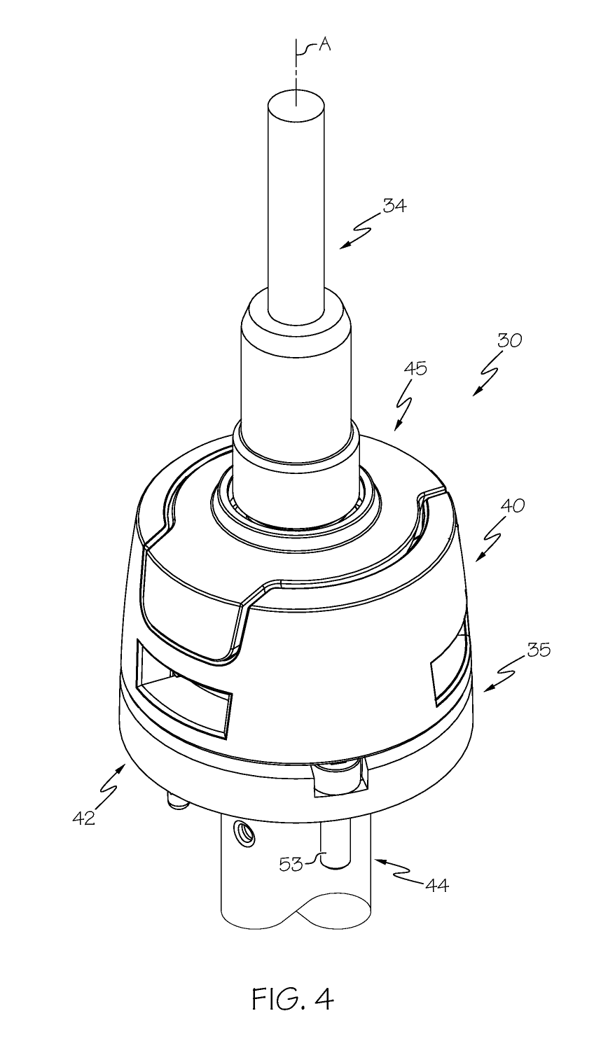

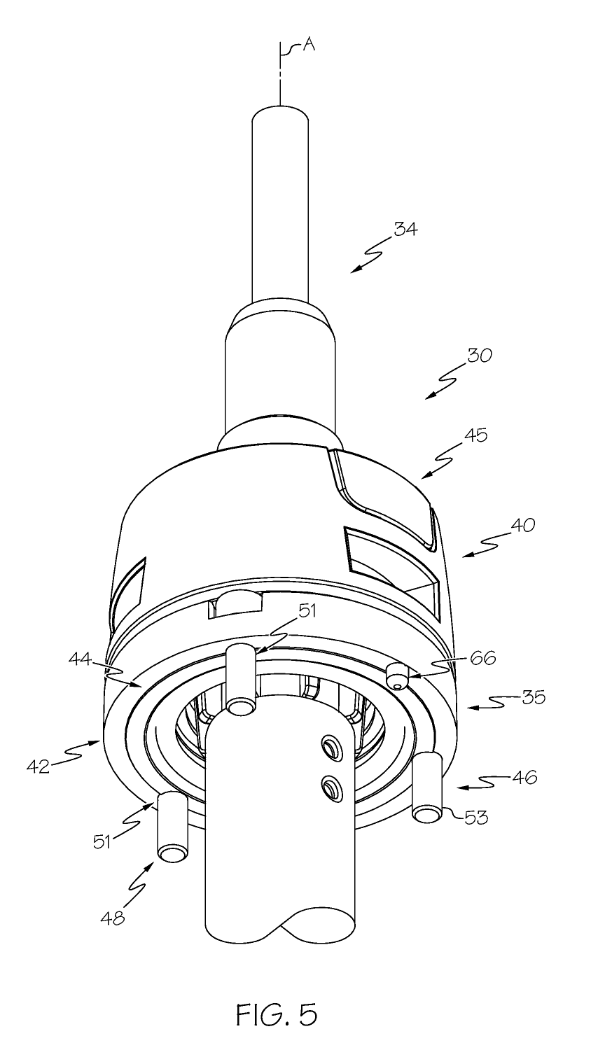

Referring to FIGS. 4 and 5, more detailed views of the lamp connector assembly 30 are illustrated. The lamp connector assembly 30 includes the sealing collar 35 and a lamp collar 40 that is rotatably and releasably connected to the sealing collar 35. The sealing collar 35 includes a base portion 42 that includes fastening locations 44, 46 and 48, each including a mounting opening 51 that is sized to receive a respective fastener 53 therethrough to connect the sealing collar to the front headplate 26. In some embodiments, the fastening locations 44, 46 and 48 are positioned asymmetrically about the perimeter of the perimeter of the base portion 42. In other words, the fastening locations 44, 46 and 48 may not be equidistant from one another about the perimeter of the base portion 42. Such an asymmetric arrangement of the fastening locations 44, 46 and 48 about the perimeter of the base portion 42 can provide a predetermined orientation of the sealing collar 35 and the UV lamp to the front headplate 26. In some embodiments, a locating pin 66 may determine orientation.

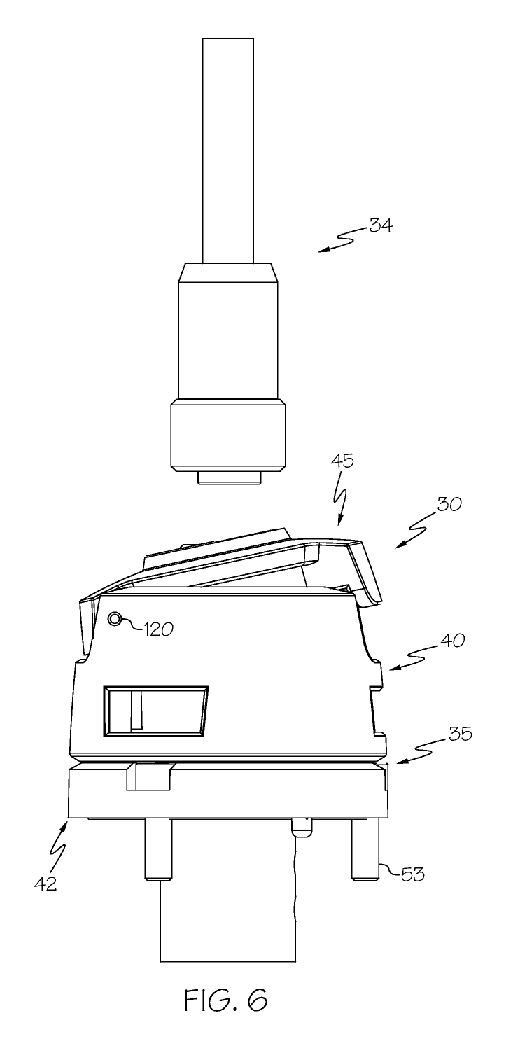

A release cap member 45 is pivotally connected to the lamp collar 40. The release cap member 45 has a pivot axis that can be orthogonal to a rotational axis A of the lamp collar 40 about the sealing collar 35. In other embodiments, the release cap member 45 may have a pivot axis that is not orthogonal to the rotational axis A of the lamp collar 40. The release cap member 45 has a locked configuration (shown in FIGS. 4 and 5) and an unlocked configuration shown in FIG. 6. As will be described in greater detail below, with the release cap member 45 in the locked configuration, rotation of the lamp collar 40 relative to the sealing collar 35 is prevented to inhibit removal of the UV lamp from the flow control structure 12 and, with the release cap member 45 in the unlocked configuration, rotation of the lamp collar 40 relative to the sealing collar 35 is allowed to allow removal of the UV lamp from the flow control structure 12, e.g., for a maintenance operation.

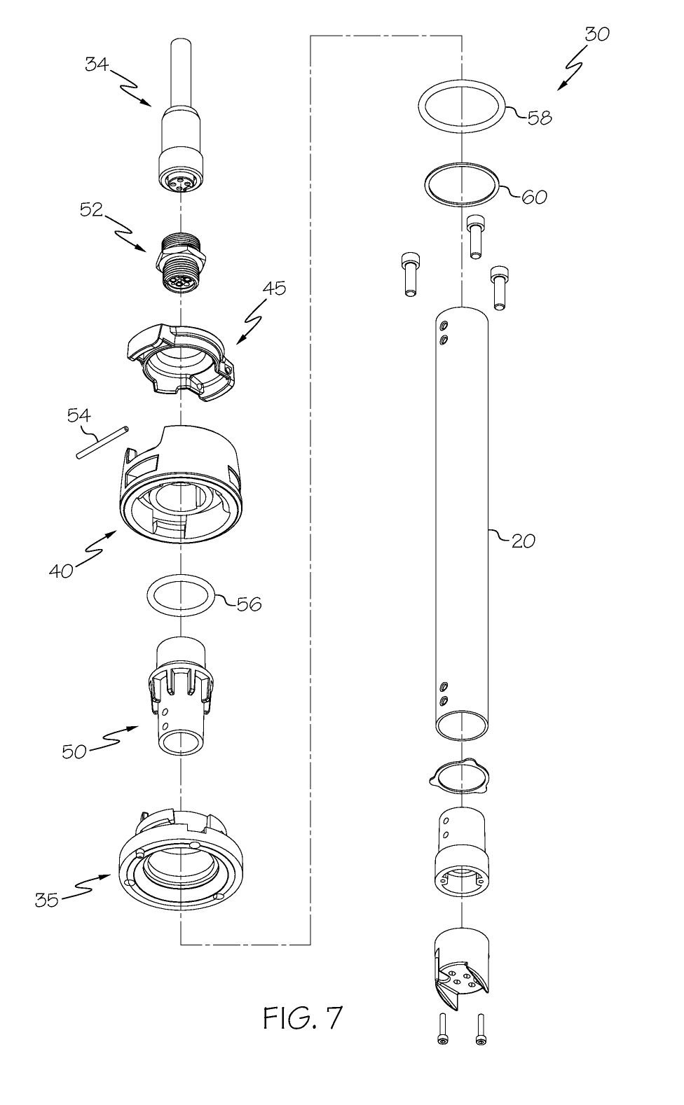

Referring to FIG. 7, an exploded view of the lamp connector assembly 30 is illustrated including the lamp collar 40, the sealing collar 35 and the release cap member 45. The lamp connector assembly 30 can further include a sleeve connector member 50 that can be used to connect and align the sleeve 20 with the sealing collar 35 and lamp collar 40. A plug adapter member 52 may be provided that provides an electrical connection between the electrical connector 34 and the UV lamp. A pivot rod 54 may be provided that provides the pivot axis for the release cap member 45 relative to the lamp collar 40, thereby facilitating movement of the release cap member 45 between the locked and unlocked configurations. One or more seals 56 (e.g., o-rings) may be provided between the lamp collar 40 and the sleeve connector member 50 to provide a water-tight seal therebetween and one or more seals 58 and 60 may be provided between the sealing collar 35 and the front headplate 26 (FIG. 2) to provide a water-tight seal therebetween.

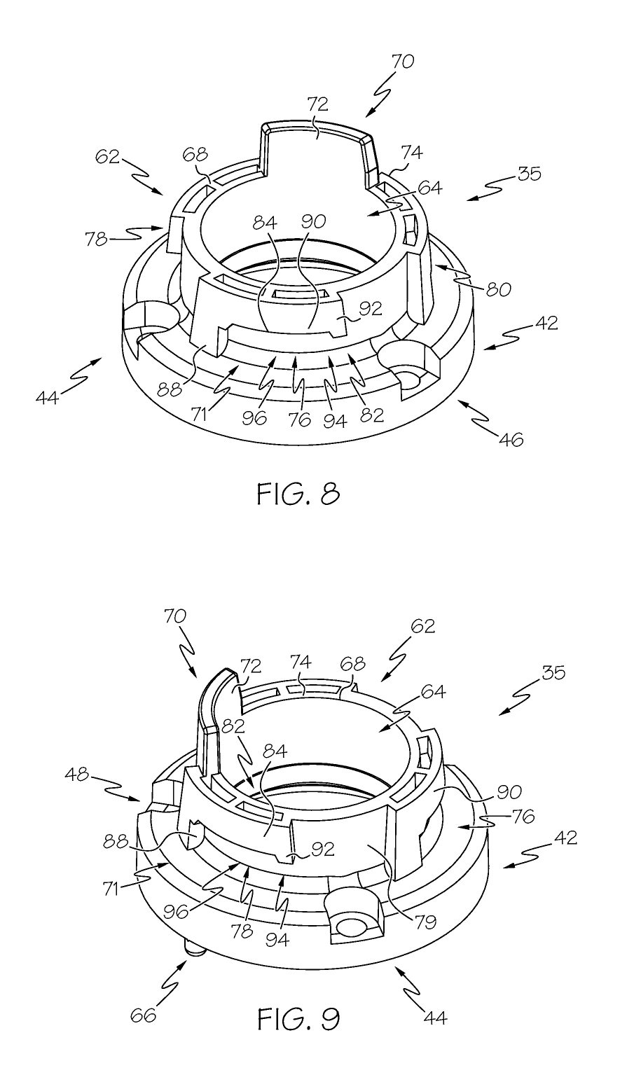

FIGS. 8 and 9 illustrate the sealing collar 35 in isolation. The sealing collar 35 may be generally cylindrical in shape and includes the base portion 42 and an interlock portion 62 that extends axially outward from the base portion 42. A lamp removal opening 64 extends through both the interlock portion 62 and the base portion 42 to provide a passageway through which the UV lamp may be removed from the flow control structure 12 (FIG. 1). The base portion 42 includes the fastening locations 44, 46 and 48, as described above (FIGS. 4 and 5). In some embodiments, the base portion 42 may also include a guide pin 66 that can be received within a recess of the front headplate 26 to orient and align the sealing collar 35 relative to the front headplate 26.

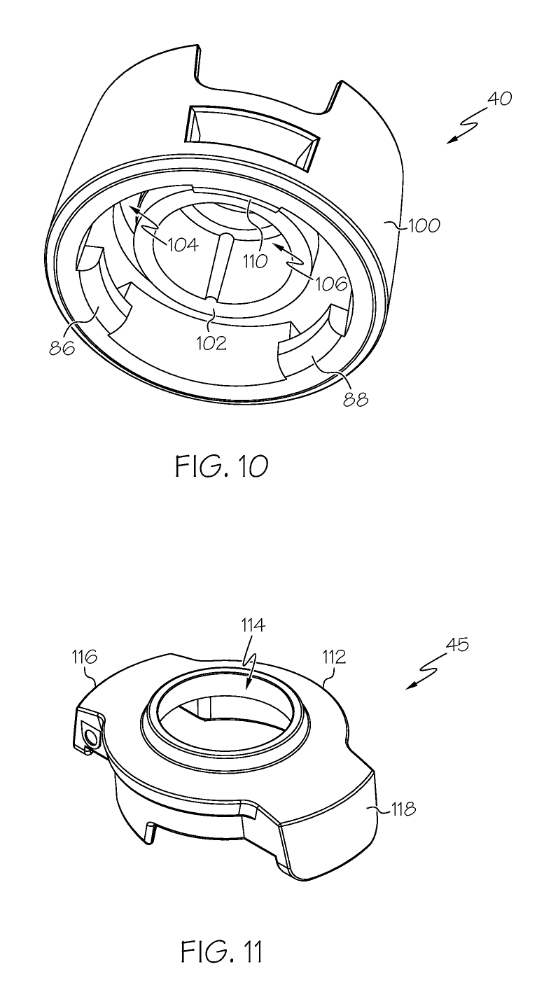

The interlock portion 62 includes a generally cylindrical wall 68 that extends axially along a central axis of the lamp removal opening 64 outward from the base portion 42. The interlock portion 62 further includes a first lamp collar interlock feature 70 including a ramp member 72 that extends axially outward beyond an outer end 74 of the wall 68. As described below, the ramp member 72 can interlock with the release cap member 45 to inhibit rotation of the lamp collar 40 relative to the sealing collar 35. The interlock portion 62 further includes a second lamp collar interlock feature 71 including interlocking slot features 76, 78 and 80 formed on an outer surface 79 of the wall 68. The interlocking slot features 76. 78 and 80 each include an arm portion 82 having an overhang surface 84 that overhangs the base portion 42 thereby forming a recess 96 that can receive a locking blade 86, 108, 110 of the lamp collar 40 (FIG. 10). The arm portions 82 each further include an axial extending stop portion 88, a circumferentially extending guide portion 90 and an axial extending interlock portion 92 that terminates at a location spaced from the base portion 42 thereby providing an entrance 94 to the recess 96 for the locking blade 86, 108, 110 of the lamp collar 40 (FIG. 10). As described below, the interlock portion 92 can inhibit rotation of the lamp collar 40 relative to the sealing collar 35 under certain pressure conditions (e.g., due to a rupture of the sleeve 36) where the lamp collar 40 is biased away from the fixed sealing collar 35 connected to the front headplate 26.

Referring to FIG. 10, the lamp collar 40 is illustrated in isolation and includes a generally cylindrical outer wall 100 and a generally cylindrical inner wall 102 that is spaced radially from the outer wall 100 thereby providing a gap 104 therebetween. The gap 104 is sized to at least partially receive the ramp member 72 to interlock with the release cap member 45 with the release cap member 45 in the locked configuration. An opening 106 is provided by the inner wall 102 that is sized to at least partially receive the plug adapter member 52 (FIG. 7) for connection with the electrical connector 34 (FIG. 2). Locking blades 86, 108 and 110 extend axially inward from the outer wall 100 for engagement with the interlocking slot features 76, 78 and 80, which can inhibit over rotation and axial movement of the lamp collar 40 relative to the sealing collar 35.

Referring to FIG. 11, the release cap member 45 is illustrated in isolation and includes a generally cylindrical body portion 112 with an opening 114 extending therethrough that is sized to at least partially receive the plug adapter member 52 (FIG. 7) for connection with the electrical connector 34 (FIG. 2). The release cap member 45 further includes opposite wing portions 116 and 118 that extend both radially outwardly from the body portion 112 and axially outward away from the body portion 112 that facilitate manual handling and changing of the release cap member 45 between the locked and unlocked configurations. Wing portion 118 may include openings 120 (FIG. 6) that are sized to pivotally receive the pivot rod 54 (FIG. 7) and pivotally connect the release cap member 45 to the lamp collar 40.

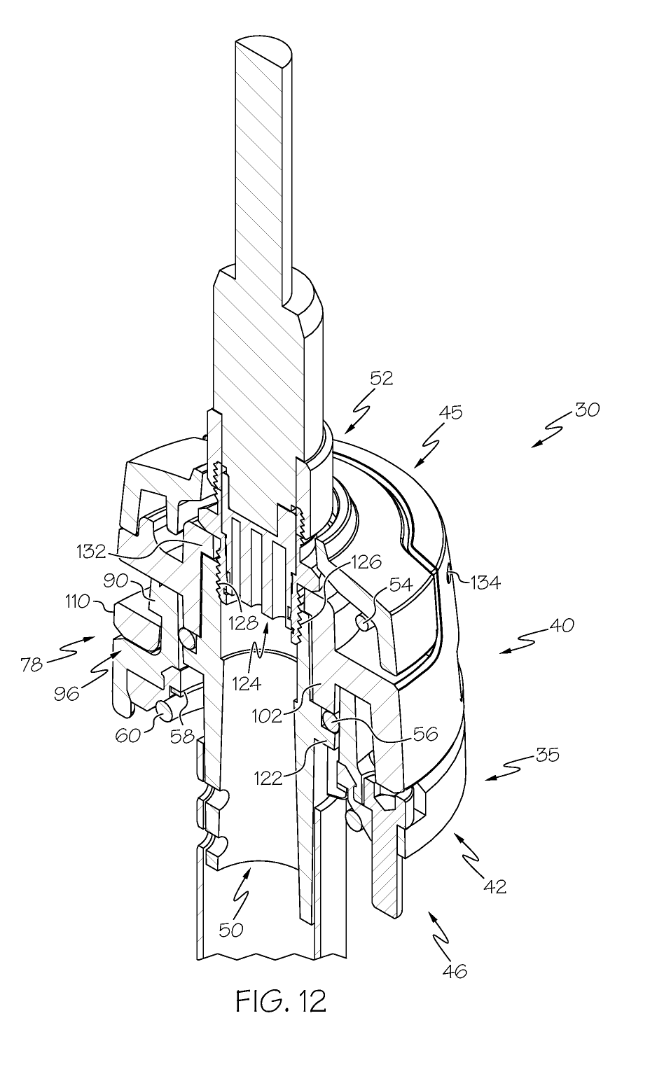

Referring to FIG. 12, additional details of the lamp connector assembly 30 including the lamp collar 40, the sealing collar 35 and release cap member 45 are shown. In FIG. 12, the lamp connector assembly 30 is assembled and shown in section in the locked configuration. As can be seen, the base portion 42 can be connected to the front headplate 26 via the fastening locations 44, 46, 48. The seal 60 may be provided between the sealing collar 35 and the front headplate 26 (FIG. 2) to provide a water-tight seal therebetween. The lamp collar 40 is releasably connected to the sealing collar 35 via the interlocking slot features 76, 78, 80. The locking blades 86, 108, 110 are received within the recesses 96 and the guide portions 90 inhibit axial movement of the lamp collar 40 relative to the sealing collar 35 without rotating the locking blades 86, 108, 110 out of the recesses 96 of the interlocking slot features 76, 78, 80. The seal 56 may be provided between the inner wall 102 of the lamp collar 40 and a radially extending flange 122 of the sleeve connector member 50 and the inner wall of the sealing collar 35. The plug adapter member 52 may be used to threadably connect the lamp collar 40 and the sleeve connector member 50. The plug adapter member 52 includes a lamp engaging end 124 having a threaded outer surface 126 that threadably connects to a threaded inner surface 128 of the sleeve connector member 50. The plug adapter member 52 further includes a radially extending flange 130 that sandwiches an inner radially extending wall 132 of the lamp collar 40 therebetween. The release cap member 45 is pivotally connected to the lamp collar 40 by the pivot rod 54 that is also received within openings 134 through the outer wall 100 of the lamp collar 40. In operation, the relative sizes on the opening 114 through the release cap member 45 and the electrical connector 34 prevent movement of the release cap member 45 from the locked position shown to the unlocked position.

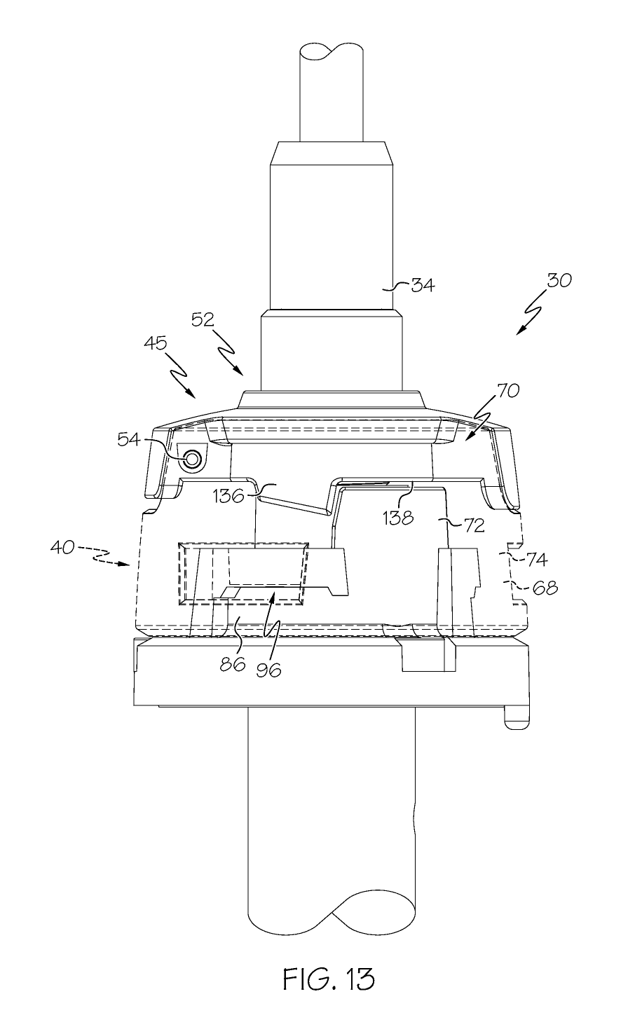

FIG. 13 illustrates the lamp connector assembly 30 with the lamp collar 40 being semi-transparent to illustrate features of the first lamp collar interlock feature 70. The first lamp collar interlock feature 70 includes the ramp member 72 that extends radially outward beyond the outer end 74 of the wall 68. The release cap member 45, which is pivotally connected to the lamp collar 40 by the pivot rod 54, includes a tab member 136 that extends outwardly from an edge 138 of the release cap member 45. In the locked configuration shown by FIG. 13, the ramp member 72 interferes with movement of the tab member 136. The locking blade 86, 108, 110 is received within the interlocking slot feature 76, 78 and 80. As can be seen, the electrical connector 34 prevents pivoting of the release cap member 45 from the locked configuration to the unlocked configuration.

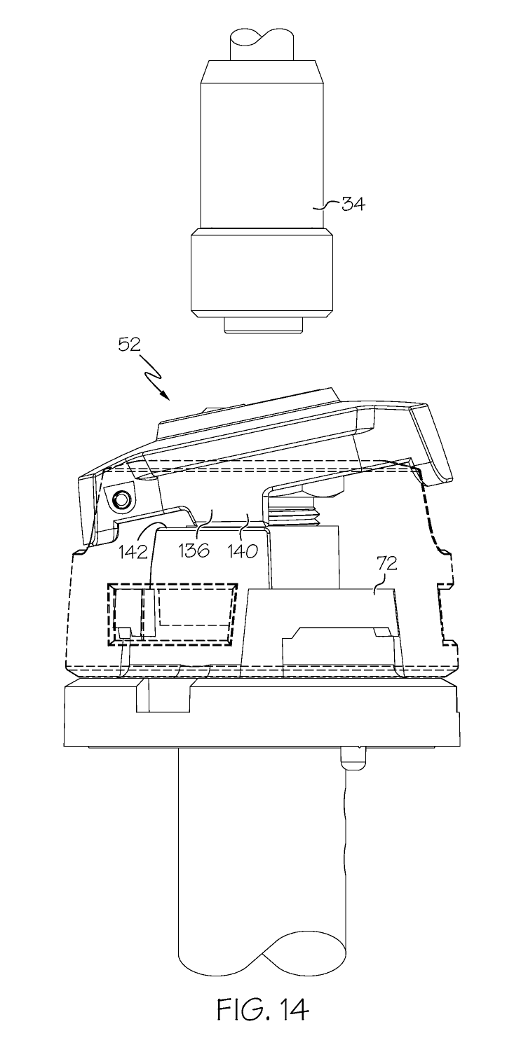

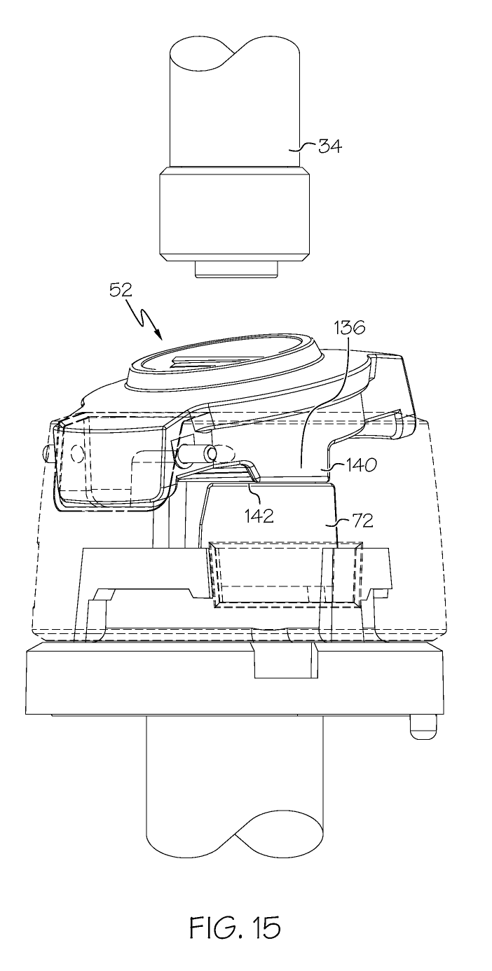

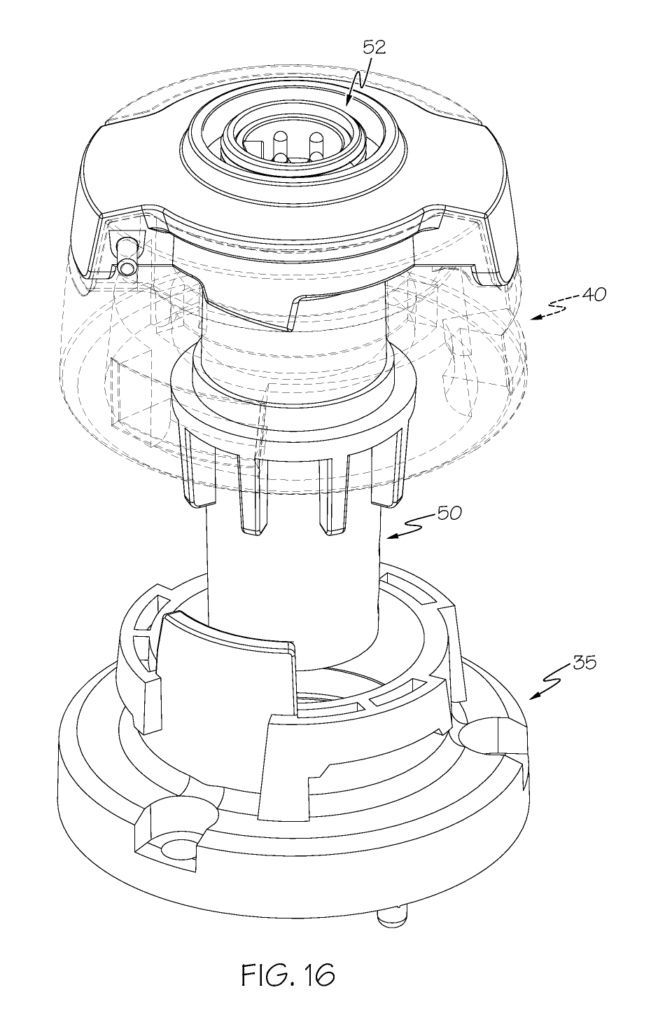

Referring to FIGS. 14 and 15, removal of the electrical connector 34 from the plug adapter member 52 allows movement of the release cap member 45 from the locked configuration to the unlocked configuration. In the unlocked configuration, the tab member 136 has an edge 140 that is shaped and arranged to rotate by an edge 142 of the ramp member 72. However, rotation of the lamp collar 40 can only be accomplished after removal of the electrical connector 34 from the plug adapter member 52, which disconnects power to the UV lamp. FIG. 16 illustrates removal of the lamp collar 40 from the sealing collar 35. As can be seen, the sleeve connector member 50 (and UV lamp) move with the lamp collar 40 when removed from the sealing collar 35.

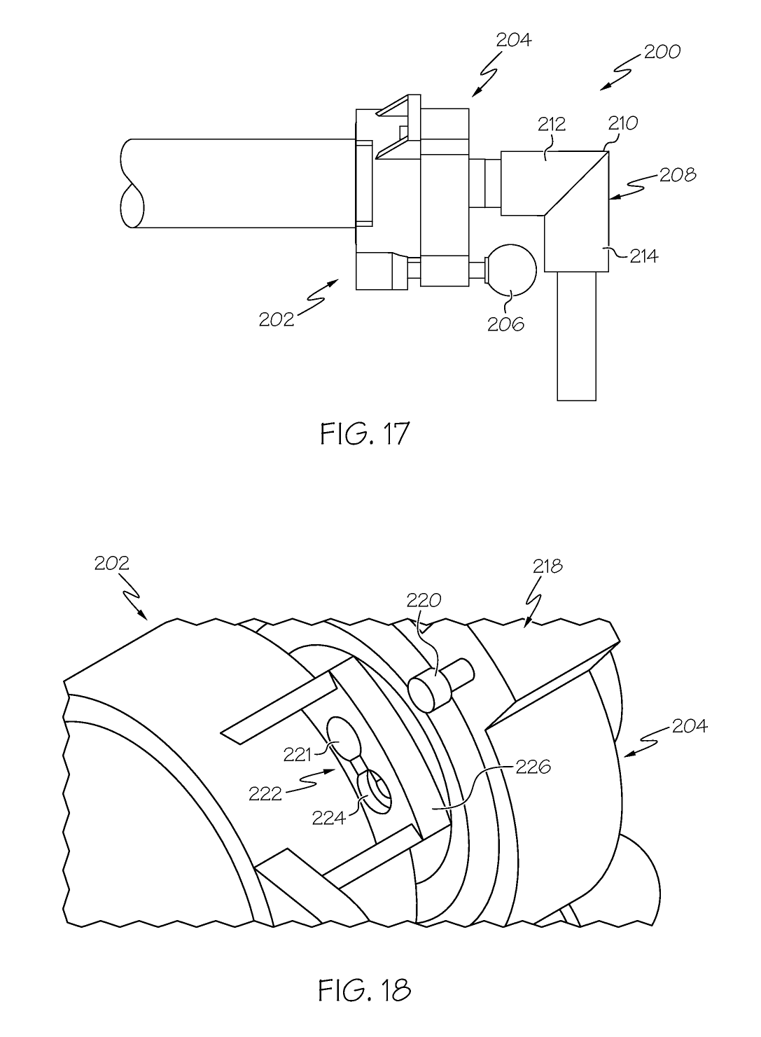

Referring now to FIG. 17, another embodiment of a lamp connector assembly 200 is illustrated. The lamp connector assembly 200 includes a sealing collar 202 and a lamp collar 204 that is rotatably connected to the sealing collar 202. A release knob member 206 is provided that inhibits rotation of the lamp collar 204 relative to the sealing collar 202 in a locked configuration. In this embodiment, an electrical connector 208 has an L-shape with a 90 degree bend 210 providing a connecting portion 212 and a bend portion 214 that is offset angularly from the connecting portion 212. Reduced clearance is provided between the release knob member 206 and the bend portion 214 of the electrical connector 208, which inhibits movement of the release knob member 206 from the illustrated locked configuration to an unlocked configuration, without disconnecting the electrical connector 208 from a plug adapter member 216.

FIG. 18 shows a more detailed view of the lamp connector assembly 200. In this view, the lamp collar 204 is released from the sealing collar 202. An engagement member 218 includes an enlarged head portion 220 that is sized to be received within a receiving portion 221 of a keyhole slot 222 provided within a flange 226 of the sealing collar 202. Once received within the receiving portion 221, the lamp collar 204 can be rotated to the locked configuration with the enlarged head portion 220 received within a retaining portion 224 of the keyhole slot 222. In some embodiments, the engagement member 218 may be spring-biased toward an extended position.

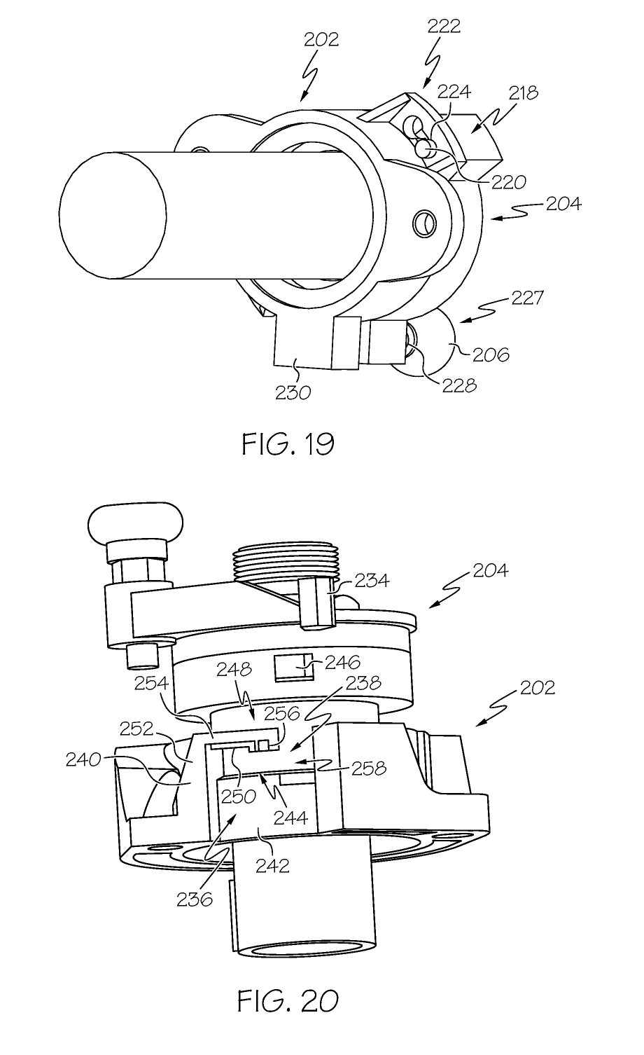

FIG. 19 illustrates the engagement member 218 in the locked configuration with the enlarged head portion 220 in the retaining portion 224 of the keyhole slot 222. The release knob member 206 includes a handle portion 227 and a pin portion 228. The release knob member 206 may be spring-biased to the extended position to engage the sealing collar 202 within an aperture provided in a ramp member 230. The release knob member 206 can prevent rotation of the lamp collar 204 with the release knob member 206 in the locked configuration. Additionally, the engagement member 218, when received by the retaining portion 224 of the keyhole slot 222, inhibits axial movement of the lamp collar 204 relative to the sealing collar 202 under pressure conditions, for example, due to a rupture of a sleeve where the lamp collar 204 is biased away from the fixed sealing collar 202 connected to the front headplate. The enlarged head portion 220 also inhibits rotation of the lamp collar 204 relative to the sealing collar 202 when the lamp collar 204 is biased away from the sealing collar 202 under the pressure conditions by recessing into the retaining portion 224 of the keyhole slot 222.

Referring to FIG. 20, the lamp collar 204 is released from the sealing collar 202. The lamp collar 204 includes a pair of tabs 234 that can be used to manually rotate the lamp collar 204 relative to the sealing collar 202. Another lamp collar interlock feature 236 includes an interlocking slot feature 238 formed in an outer wall 240. The interlocking slot feature 238 includes an arm portion 248 having an overhang surface 250 that overhangs a base portion 242 thereby forming a slot 244 that can receive a locking blade 246 of the lamp collar 204. The arm portion 248 further includes an axial extending stop portion 252, a circumferentially extending guide portion 254 and an axial extending interlock portion 256 that terminates at a location spaced from the base portion 242 thereby providing an entrance 258 to the slot 244 for the locking blade 246 of the lamp collar 204. As above, the interlock portion 256 can inhibit rotation of the lamp collar 204 relative to the sealing collar 202 under certain pressure conditions (e.g., due to a rupture of the sleeve) where the lamp collar 204 is biased away from the fixed sealing collar 202 connected to the front headplate.

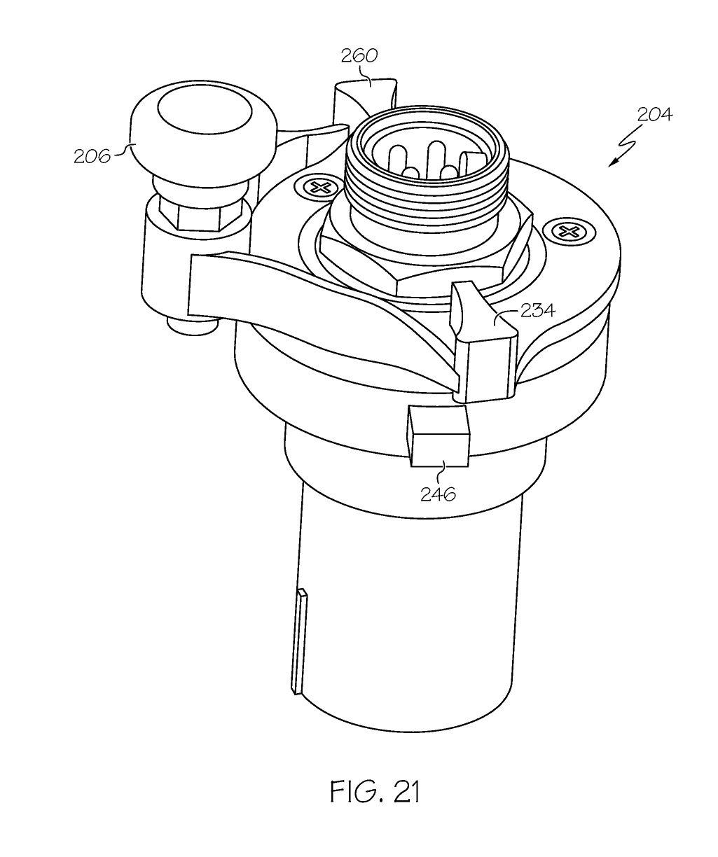

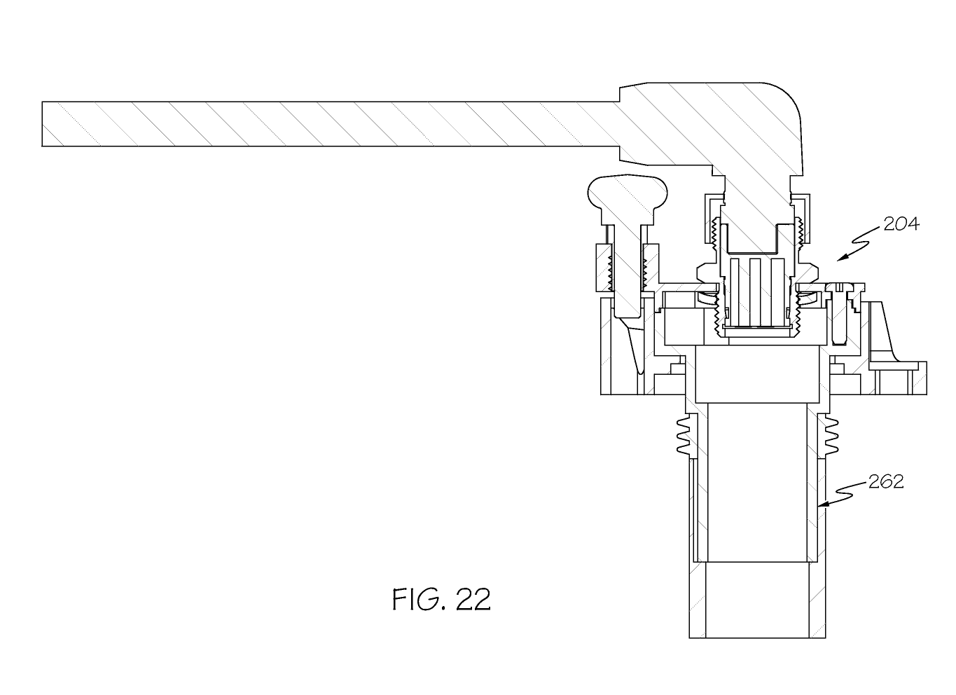

Referring to FIG. 21, an elevated, perspective view of the lamp collar 204 is illustrated including the tabs 234 and 260, locking blade 246 and release knob member 206. FIG. 22 illustrates a section view of the lamp collar 204. As can be seen, in some embodiments, a sleeve connector member 262 may be formed integrally with the lamp collar 204 as opposed to being a separate member.

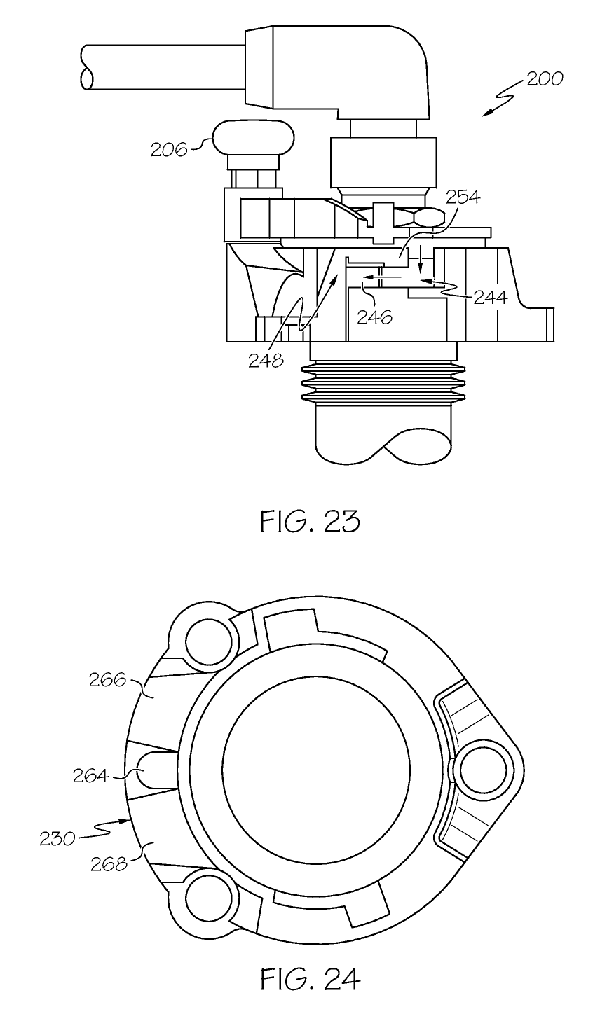

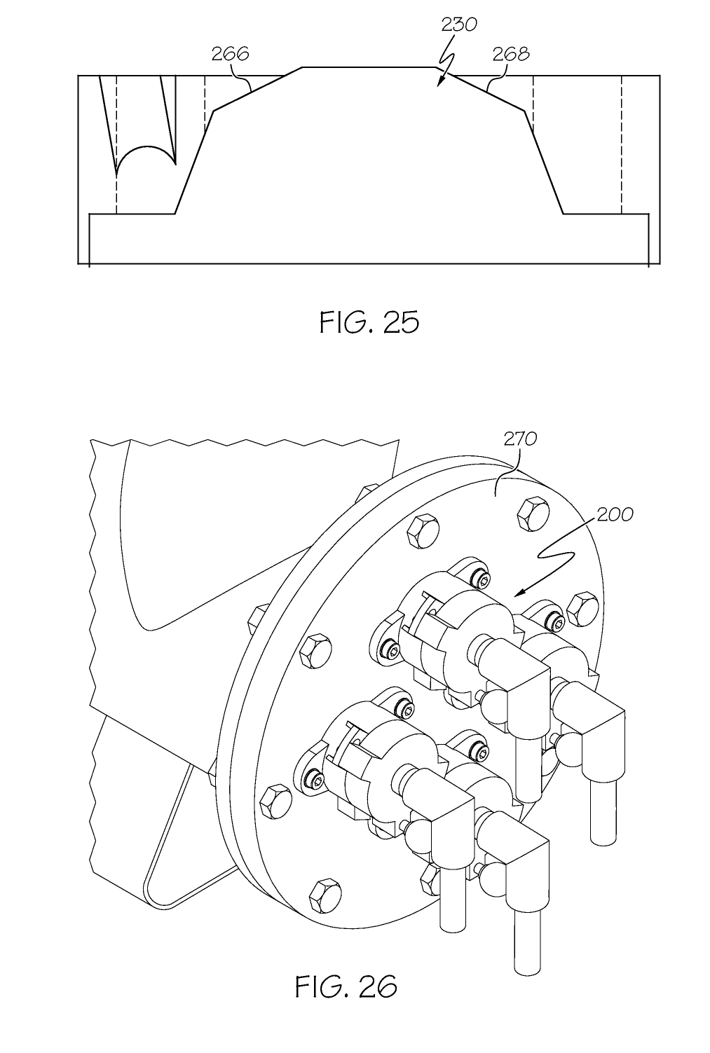

Referring to FIG. 23, the lamp connector assembly 200 is illustrated in the locked configuration with the locking blade 246 received in the slot 244, below the guide portion 254 of the arm portion 248. The release knob member 206 is received in the aperture 264 of the ramp member 230 (FIG. 24). FIG. 25 illustrates a side view of the ramp member 230. The ramp member 230 includes angles surfaces 266 and 268 that allow the release knob member 206 to slide along and lock into position. FIG. 26 illustrates the lamp connector assemblies 200 connected to the front headplate 270.

For the purposes of describing and defining the present invention it is noted that the terms “substantially” and “approximately” are utilized herein to represent the inherent degree of uncertainty that may be attributed to any quantitative comparison, value, measurement, or other representation. The terms “substantially” and “approximately” are also utilized herein to represent the degree by which a quantitative representation may vary from a stated reference without resulting in a change in the basic function of the subject matter at issue.

Having described the subject matter of the present disclosure in detail and by reference to specific embodiments thereof, it will be apparent that modifications and variations are possible without departing from the scope of the invention defined in the appended claims.

1. A lamp connector assembly for an ultraviolet (UV) water treatment vessel, the lamp connector assembly comprising:
a sealing collar configured to connect to a headplate of the UV treatment vessel, the sealing collar comprising a first lamp collar interlock feature;
a lamp collar configured to releasably connect to the sealing collar; and
a release member moveably connected to the lamp collar, the release member having a locked configuration that engages the first lamp interlock feature to prevent movement of the lamp collar relative to the sealing collar and an unlocked configuration that disengages the first lamp interlock feature to allow movement of the lamp collar relative to the sealing collar.

2. The lamp connector assembly of claim 1, wherein the release member comprises a releasable cap member that is pivotally connected to the lamp collar.

3. The lamp connector assembly of claim 1, wherein the first lamp collar interlock feature includes a ramp member that extends into the lamp collar.

4. The lamp connector assembly of claim 3, wherein the release cap member further includes a tab member that extends into the lamp collar for engaging the ramp member with the release cap member in the locked configuration.

5. The lamp connector assembly of claim 4, wherein the release cap member includes an opening to receive a plug adapter member, wherein movement of the release cap member from the locked configuration to the unlocked configuration is prevented with an electrical connector received by the plug adapter member.

6. The lamp connector assembly of claim 1, wherein the sealing collar comprises a second interlock feature including an interlocking slot feature that matingly receives a locking blade of the lamp collar therein with the release member in the locked configuration.

7. The lamp connector assembly of claim 1, wherein the release member comprises a release knob member moveably connected to the lamp collar having a pin portion that is sized to be received within an aperture provided by the sealing collar.

8. The lamp connector assembly of claim 1, wherein the lamp member comprises an engagement member including an enlarged head portion that is sized to be received by a keyhole slot provided by the sealing collar to inhibit axial movement of the lamp collar away from the sealing collar.

9. An ultraviolet (UV) treatment vessel comprising:
a flow control structure comprising an inlet port for ingress of untreated water into a treatment chamber and an outlet port for egress of treated water from the treatment chamber;
a lamp connector assembly connected to a headplate of the flow control structure, the lamp connector assembly comprising:
a sealing collar connected to the headplate of the UV treatment vessel, the sealing collar comprising a first lamp collar interlock feature;
a lamp collar configured to releasably connect to the sealing collar; and
a release member moveably connected to the lamp collar, the release member having a locked configuration that engages the first lamp interlock feature to prevent movement of the lamp collar relative to the sealing collar and an unlocked configuration that disengages the first lamp interlock feature to allow movement of the lamp collar relative to the sealing collar.

10. The UV treatment vessel of claim 9, wherein the release member comprises a releasable cap member that is pivotally connected to the lamp collar.

11. The UV treatment vessel of claim 9, wherein the first lamp collar interlock feature includes a ramp member that extends into the lamp collar.

12. The UV treatment vessel of claim 11, wherein the release cap member further includes a tab member that extends into the lamp collar for engaging the ramp member with the release cap member in the locked configuration.

13. The UV treatment vessel of claim 12, wherein the release cap member includes an opening to receive a plug adapter member, wherein movement of the release cap member from the locked configuration to the unlocked configuration is prevented with an electrical connector received by the plug adapter member.

14. The UV treatment vessel of claim 9, wherein the sealing collar comprises a second interlock feature including an interlocking slot feature that matingly receives a locking blade of the lamp collar therein with the release member in the locked configuration.

15. The UV treatment vessel of claim 9, wherein the release member comprises a release knob member moveably connected to the lamp collar having a pin portion that is sized to be received within an aperture provided by the sealing collar.

16. The UV treatment vessel of claim 15 further comprising a plug adapter member located within an opening of the lamp collar and an electrical connector including a connecting portion that connects to the plug adapter member and a bend portion that overhangs the release knob member to inhibit movement of the release knob member out of the aperture provided by the sealing collar with the connecting portion connected to the plug adapter member.

17. The UV treatment vessel of claim 9, wherein the lamp member comprises an engagement member including an enlarged head portion that is sized to be received by a keyhole slot provided by the sealing collar to inhibit axial movement of the lamp collar away from the sealing collar.

18. A lamp connector assembly for an ultraviolet (UV) water treatment vessel, the lamp connector assembly comprising:
a sealing collar configured to connect to a headplate of the UV treatment vessel, the sealing collar comprising a first lamp collar interlock feature;
a lamp collar configured to releasably connect to the sealing collar, the first lamp collar interlock feature inhibiting rotation of the lamp collar relative to the sealing collar in a locked configuration; and
the sealing collar comprising a second interlock feature including an interlocking slot feature that matingly receives a locking blade of the lamp collar therein with the release member in the locked configuration to inhibit axial movement of the lamp collar away from the sealing collar.

19. The lamp connector assembly of claim 18, wherein the interlocking slot features includes an arm portion that overhangs a base portion of the sealing collar thereby forming a recess that receives the locking blade of the lamp collar.

20. The lamp connector assembly of claim 18 further comprising a release member moveably connected to the lamp collar, the release member engages the first lamp interlock feature to prevent movement of the lamp collar relative to the sealing collar in the locked configuration and disengages the first lamp interlock feature to allow movement of the lamp collar relative to the sealing collar in the unlocked configuration.
